SEQUENCE LISTING (6 sequences)

SEQ ID NO: 1 - DNA, 3191 bp, Homo sapiens
ccggctggaggagctgaggttccgagtgcggccgctgctgggctggcgggcgggcagagcacggcaccctggcagcagggcccacgccacggggccatgggcagctcgagccaggcaggctgctgcccacgcttactgccagggtgaccccagccctggggcccagccacaaccaccctggcttcatgccaggggctgctctggttgccagtcggccagcctcgggggtgcagcctgggctgggactgctgctggggtgcaggtgaggcagtggccgggccctcaggccccagggcaggcaggctgcagggagccaagtcctccatggcggccccagccgggaaggcgagcaggacaggggctctgcggcccagggcccagaaaggccgggtgaggcgggccgtgcgcatctccagcctcgtggcccaggaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttagtggggctgcccagtgtggacacgtggctcacccagggatcaaggcgctgctgggccgctccccttggaggccctgctcaggaggccctgaccgtggaaggggagaggaactcgaaagcacagctcctcccccagcccttgggaccatcttctcctgcagtcccctgggccccagtgagaggggcaggggcagggccggcagtaggtggggcctgtggtccccccactgccctgagggcattagctggtctaactgcccggaggcacccggccctgggccttaggcacctcaaggacttttctgctatttactgctcttattgttaaggataataattaaggatcatatgaataattaatgaagatgctgatgactatgaataataaataattatcctgaggagaaaa

SEQ ID NO: 2 - protein, 819 aa, Homo sapiens
MAAPAGKASRTGALRPRAQKGRVRRAVRISSLVAQEVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 3 - protein, 36 aa, Homo sapiens
MAAPAGKASRTGALRPRAQKGRVRRAVRISSLVAQE

SEQ ID NO: 4 - protein, 36 aa, Mus musculus
MAIPAGKESRTGALQPRAQKGRVRRAVRISSLVAQE

SEQ ID NO: 5 - protein, 36 aa, Rattus norvegicus
MAIPAGKESRTGALQTRAQKGRVRRAVRISSLVAQE

SEQ ID NO: 6 - protein, 36 aa, Canis lupus
MAAPAGKASRTGALQPRAQKGRVRRAVRISSLVAQE

(19) United States
(12) Patent Application Publication
(10) Pub. No.: US 2010/0003690 A1
(43) Pub. Date: Jan. 7, 2010

(54) COMPOSITIONS AND METHODS FOR DISCOVERING AGENTS FOR PHARMACOLOGICAL RESCUE OF HERG 1B SUBUNITS FOR THE TREATMENT OF LONG QT SYNDROME

(76) Inventors: Gail A. Robertson, Madison, WI (US); Michael J. Ackerman, Rochester, MN (US); Pallavi Phartiyal, Washington, DC (US)

Correspondence Address: QUARLES & BRADY LLP, 411 E. WISCONSIN AVENUE, SUITE 2040, MILWAUKEE, WI, 53202-4497, US

(21) Appl. No.: 12/490,987

(22) Filed: Jun. 24, 2009

Related U.S. Application Data
(60) Provisional application No. 61/075,244, filed on Jun. 24, 2008.

(51) Int. Cl. C12Q 1/68; C12N 5/06; C12N 5/08; C40B 30/04

(52) U.S. Cl. 435/6; 435/358; 435/369; 435/365; 506/9

(57) ABSTRACT

Cells engineered to express a detectable hERG 1b polypeptide and methods for identifying agents that facilitate hERG 1b maturation using such cells are provided. A method for analyzing a biological sample for the presence of an amino acid substitution at position 8 of the hERG 1b N-terminus is further provided.

### CROSS-REFERENCE TO RELATED APPLICATIONS

This application claims the benefit of U.S. Provisional Patent Application No. 61/075,244, filed Jun. 24, 2008, incorporated herein by reference as if set forth in its entirety.

### STATEMENT REGARDING FEDERALLY SPONSORED RESEARCH OR DEVELOPMENT

This invention was made with United States government support awarded by the following agency: National Institutes of Health, HL68868 and HL081780. The United States government has certain rights in this invention.

### BACKGROUND

Inwardly-rectifying, repolarizing potassium (I_{Kr}) channels encoded by the human ether-a-go-go-related gene (hERG) contain two alpha subunits—a hERG 1a subunit and a hERG 1b subunit hERG channels contribute to the final phase of an action potential that returns a cell to its resting state.

hERG 1a and hERG 1b, produced from alternative transcripts of the hERG1 gene (see, GenBank Accession Nos. CS114788 and CS114790, GenBank Accession Nos. NM_{—}000238 and NM_{—}172057), have identical transmembrane and carboxy-terminal sequences, but divergent amino termini. hERG 1a and hERG 1b interact to promote heteromeric assembly in early I_{Kr} channel biogenesis. While heteromeric hERG 1a/1b channels and homomeric hERG 1a channels yield currents having distinct properties, homomeric hERG 1b channels produce undetectable or very little current due to retention in the endoplasmic reticulum. hERG 1b channels can be “rescued” by mutations of an endoplasmic reticulum-retention signal, and under these conditions produce currents capable of repolarization.

Perturbations of hERG channels can result in Long QT syndrome (LQTS), an inherited or acquired cardiac condition that can lead to arrhythmias and sudden cardiac death in human individuals. LQTS is characterized by a prolonged repolarization (recovery) following depolarization (excitation) of the ventricles. Individuals having LQTS exhibit a characteristic prolonged QT interval on an electrocardiogram (ECG).

LQTS can be inherited or acquired. In inherited LQTS, hERG 1a subunit loss-of-function mutations (which can be deletions, insertions or substitutions) often reside in the N terminus of hERG 1a. The mutations typically truncate the protein, alter gating properties and/or cause trafficking deficiencies. In the absence of hERG1a activity, hERG 1b is synthesized but is retained in the endoplasmic reticulum. Even in the presence of hERG 1a, some hERG 1b remains within the endoplasmic reticulum. hERG 1a/1b channels confer 80% more repolarizing charge than hERG 1a homomers when expressed in HEK-293 cells under conditions mimicking a cardiac ventricular action potential. Thus, loss of hERG 1b subunits is expected to reduce repolarizing capacity and can lead to long QT syndrome.

Acquired LQTS, which affects an estimated 1% to 8% of the general population, is caused by pharmacological agents that bind hERG channels and reduce the amplitude of repolarizing currents. At least fifty FDA-approved agents are known to bind hERG channels. About 50% of all lead compounds are eliminated from a drug-discovery process because they block hERG channels in preclinical tests. Avoiding acquired LQTS is a priority in new drug development.

For the foregoing reasons, there is a need for compositions and methods for screening for agents that promote surface expression of hERG 1b subunits.

### BRIEF SUMMARY

The invention relates generally to cells, methods and compositions for evaluating LQTS at the cellular level, and more particularly to finding agents that facilitate (i.e., enhance or stabilize) movement of an hERG 1b polypeptide subunit from the perinuclear compartment (endoplasmic reticulum and Golgi apparatus) to the plasma membrane. Such movement is referred to herein as subunit maturation, or simply as maturation.

In a first aspect, a cell engineered for screening for agents that facilitate subunit maturation contains a polynucleotide having a hERG 1b polypeptide subunit-encoding portion and an operably-linked upstream expression control sequence not endogenously linked to the polynucleotide. The cell into which the polynucleotide was introduced expresses a detectable hERG 1b polypeptide. The polynucleotide can include the nucleic acid sequence of SEQ ID NO: 1. Optionally, the polynucleotide can be operably-linked to a reporter such as to encode a hERG 1b-reporter fusion polypeptide. The fusion polypeptide can have the amino acid sequence of SEQ ID NO:2.

In certain embodiments, the cell can be a HEK-293 cell, COS-7 cell, CHO-K1 cell, L929 cell, or a Xenopus oocyte.

In certain embodiments, the reporter can be a FLAG-tag (or FLAG octapeptide) or other epitope, such as a hemagglutinin (HA)-tag, glutathione S-transferase (GST)-tag, histidine (His)-tag or c-myc-tag, that can interact with labeled antibodies, such as biotin-, fluorophore- or enzyme-labeled antibodies.

In a second aspect, a method of screening for agents that facilitate subunit maturation includes the steps of exposing a cell engineered for screening to a test agent and evaluating localization of hERG 1b in the agent-exposed cell engineered for screening relative to an untreated cell engineered for screening. If the tested agent facilitates hERG 1b maturation, hERG 1b will be localized at the cell surface of the agent-exposed cell where it can be visualized using detection technologies, preferably detection techniques relying on fluorescence or high-throughput screening enabling measurement of membrane currents. If the test agent does not facilitate hERG 1b maturation, hERG 1b will remain in the perinuclear compartment and cannot be visualized at the cell surface.

A previously uncharacterized mutation (disclosed herein) causing dramatic reduction of hERG 1b protein and perturbation of current properties was identified in a patient with long QT syndrome and represents the first example of 1b-specific long QT syndrome. Stabilization or rescue of the hERG 1b mutant protein should preserve the hERG 1a/1b interaction and help maintain normal repolarizing levels.

These and other features, objects and advantages of the present invention will become better understood from the description that follows. The description of preferred embodiments is not intended to limit the invention or to cover all modifications, equivalents and alternatives. Reference should therefore be made to the claims herein for interpreting the scope of the invention.

### BRIEF DESCRIPTION OF THE DRAWINGS

FIG. 1 depicts a Western blot analysis of cell lysates from HEK-293 cells transfected with sequences encoding for hERG 1b (lane 1); hERG 1a (lane 2); hERG 1a and 1b (lane 3); hERG 1b with an alanine-to-valine substitution (A8V) (lane 4); hERG 1a (lane 5); hERG 1a and 1bA8V (lane 6); or hERG 1b (lane 7).

FIG. 2A depicts current traces from hERG 1a stably expressed in HEK-293 cells. FIG. 2B depicts current traces from hERG 1a stably expressed in HEK-293 cells transfected with hERG 1bA8V.

### DESCRIPTION OF PREFERRED EMBODIMENTS

The present invention relates to the inventors' observation that in healthy heart tissue, hERG 1b pools in the endoplasmic reticulum and fails to mature. At or near its amino terminus, hERG 1b subunit includes an amino acid motif associated with retaining the subunit in the endoplasmic reticulum. The inventors determined that hERG 1b includes an Arg-X-Arg motif (RXR; two arginine residues separated by any single residue). Alteration of the RXR motif promoted hERG 1b export from the endoplasmic reticulum and expression of functional homomeric hERG 1b channels in the plasma membrane.

These observations suggested to the inventors that hERG 1a can facilitate hERG 1b maturation by preventing or masking RXR-mediated retention. It follows that an agent that masks hERG 1b's RXR motif can rescue hERG 1b from the endoplasmic reticulum reservoir and can bolster I_{Kr} in certain individuals, especially in individuals receiving pharmaceuticals that block hERG activity.

The discovery of a hERG 1b-specific mutation in a patient suggested to the inventors that hERG 1b protein was lost because of defective protein folding and reduced stability. It follows that an agent stabilizing the hERG 1b protein can rescue the protein and bolster I_{Kr} in individuals affected by hERG 1b mutations.

Cells suited for use in the methods of the invention preferably lack endogenous hERG channels. One such cell type is HEK-293 cells (American Type Culture Collection; ATCC; Manassas, Va.; Catalog No. CRL-1573). See also, Graham F, et al., “Characteristics of a human cell line transformed by DNA from human adenovirus type 5,” J. Gen. Virol. 36:59-74 (1977), incorporated herein by reference as if set forth in its entirety. HEK-293 cells are easy to reproduce and to maintain, are amenable to transfection using a wide variety of methods, have a high efficiency of transfection and protein production, have faithful translation and processing of proteins and have a small cell size with minimal processes appropriate for electrophysiological experiments.

Another suitable cell type is COS-7 cells (ATCC (Catalog No. CRL-1651). See also, Gluzman Y, “SV40-transformed simian cells support the replication of early SV40 mutants,” Cell 23:175-182 (1981), incorporated herein by reference as if set forth in its entirety. Like HEK-293 cells, COS-7 cells are easy to reproduce and maintain and are amenable to transfection using a wide variety of methods.

Yet another suitable cell type is Xenopus oocytes. Xenopus oocytes are commonly used for electrophysiological recordings because of their large size (˜1.0 mm), which makes their handling and manipulation easy. Xenopus oocytes are readily amenable to injection, and thus express functional ion channels when injected with cRNA for an ion channel.

The ability to determine the location of hERG 1b in the cell is facilitated by providing a fusion protein having an hERG 1b portion with a reporter portion that encodes a FLAG-, GST-, hemagglutinin (HA)-, histidine (H is)- or c-myc-tag. FLAG-tags are well known in the art and often comprise a DYKDDDDK. See, e.g., Chubet R & Brizzard B, “Vectors for expression and secretion of FLAG epitope-tagged proteins in mammalian cells,” Biotechniques 20:136-141 (1996); Knappik A & Pluckthun A, “An improved affinity tag based on the FLAG peptide for the detection and purification of recombinant antibody fragments,” Biotechniques 17:754-761 (1994); and Slootstra J, et al., “Identification of new tag sequences with differential and selective recognition properties for the anti-FLAG monoclonal antibodies M1, M2 and M5,” Mol. Divers. 2:156-164 (1997), each of which is incorporated herein by reference as if set forth in its entirety. HA tags are also well known in the art and are derived from an epitope of the influenza hemagglutinin protein, which has been extensively used as a general epitope tag in expression vectors. See, Field J, et al., “Purification of a RAS-responsive adenylyl cyclase complex from Saccharomyces cerevisiae by use of an epitope addition method,” Mol. Cell. Biol. 8:2159-2165 (1998), each of which is incorporated herein by reference as if set forth in its entirety. Furthermore, cMyc-tags are well known in the art and are derived from a human proto-oncogene. See, De Buck E, et al., “The use of the cMyc epitope tag can be problematic for protein detection in Legionella pneumophila,” J. Microbiol. Methods. 59:131-134 (2004); and Li J, et al., “Application of Microfluidic Devices to Proteomics Research,” Mol. Cell. Proteomics 1:157-168 (2002), each of which is incorporated herein by reference as if set forth in its entirety. Moreover. GST-tags and His-tags are also well known in the art. See, e.g., Smith D & Johnson K, “Single-step purification of polypeptides expressed in Escherichia coli as fusions with glutathione S-transferase,” Gene 67:31-40 (1988); Toye B, et al., “Immunologic characterization of a cloned fragment containing the species-specific epitope from the major outer membrane protein of Chlamydia trachomatis,” Infect. Immunol. 58:3909-3913 (1990); Beekman J, et al., “A rapid one-step method to purify baculovirus-expressed human estrogen receptor to be used in the analysis of the oxytocin promoter,” Gene 146:285-289 (1994); Poon R & Hunt T, “Reversible immunoprecipitation using histidine- or glutathione S-transferase-tagged staphylococcal protein A,” Anal. Biochem. 218:26-33 (1994); Hengen P, “Purification of His-Tag fusion proteins from Escherichia coli,” Trends Biochem. Sci. 20:285-286 (1995); and Dieryck W, et al., “Cloning, expression and two-step purification of recombinant His-tag enhanced green fluorescent protein over-expressed in Escherichia coli,” J. Chromatogr. B Analyt. Technol. Biomed. Life Sci. 786:153-159 (2003), each of which is incorporated herein by reference as if set forth in its entirety. The reporter portion therefore identifies the location of hERG 1b through any of various imaging techniques known to one of ordinary skill in the art (e.g., fluorescence microscopy).

Labeled antibodies for detection of surface expression of hERG subunits are well known in the art. Such labels can include, but are not limited to the following: biotin, fluorophoies (e.g., allophycocyanin (APC), the Alexa dyes (i.e., Alexa 350, Alexa 430, Alexa 488, Alexa 532, Alexa 546, Alexa 568 and Alexa 594), green fluorescent protein (GFP), fluorescein isothiocyanate (FITC), phycoerythrin (PE), cyanine-3 (Cy3), cyanine-5 (Cy5), rhodamine and Texas red), polysaccharides (e.g., dextran 250 and dextran 70) and enzymes (e.g., alkaline phosphatase and horseradish peroxidase).

The inventors specifically contemplate that the engineered cells can also express a hERG 1a subunit variant that fails to interact with the hERG 1b retention signal sequence. Since hERG 1a and hERG 1b are alternate transcripts produced by the HERG1 gene, a mutation in an exon that encodes the hERG 1a N-terminus is unlikely to affect hERG 1b expression. Suitable mutant hERG 1a subunits include, but are not limited to, those disclosed in publicly available databases provided by, for example, the Working Group on Arrhythmias of the European Society of Cardiology or the Statens Serum Institute, each of which is available on the World Wide Web. In addition, mutant hERG 1a subunits suitable for use herein are shown in Table 1.

TABLE 1
HERG 1a mutations.

| | | Coding | | Ethnic | |
|---|---|---|---|---|---|
| Nucleotide change | Mutation | effect | Region | back-ground | Reference |
| — | S26I | Missense | PAS domain | USA | Moss et al, 2002 |
| 87C > A | F29L | Missense | PAS domain | USA | Chen et al, 1999, |
| | | | | | Splawski et al, 2000 |
| 98A > C | N33T | Missense | PAS domain | USA | Chen et al, 1999, |
| | | | | | Splawski et al, 2000 |
| 132C > A | C44X | Missense | PAS domain | USA | Splawski et al, 2000 |
| 140G > T | G47V | Missense | PAS domain | USA | Splawski et al, 2000 |
| 157G > C | G53R | Missense | PAS domain | USA | Chen et al, 1999, |
| | | | | | Splawski et al, 2000 |
| 167G > A | R56Q | Missense | PAS domain | USA | Chen et al, 1999, |
| | | | | | Splawski et al, 2000 |
| 196T > G | C66G | Missense | PAS domain | USA | Chen et al, 1999, |
| | | | | | Splawski et al, 2000 |
| 209A > G | H70R | Missense | PAS domain | USA | Chen et al, 1999, |
| | | | | | Splawski et al, 2000 |
| 215C > A | P72Q | Missense | PAS domain | USA | Splawski et al, 2000 |
| 221-251del | 31 bp deletion | Frameshift | PAS domain | USA | Splawski et al, 2000 |
| 234-250dup | 16 bp duplication | Frameshift | PAS domain | USA | Splawski et al, 2000 |
| — | A78P | Missense | PAS domain | USA | Moss et al, 2002 |
| 241C > T | Q81X | Nonsense | Pas domain | USA | Splawski et al, 2000 |
| 244-252 ins9 | 82-84insIAQ | Duplication | PAS-domain | Da | Larsen et al, 2001 |
| — | fs83/37aa | Frameshift | PAS domain | USA | Moss et al, 2002 |
| 257G > T | L86R | Missense | PAS domain | USA | Chen et al, 1999, |
| | | | | | Splawski et al, 2000 |
| 260T > C | L87P | Missense | PAS domain | Du | Jongbloed et al, 2002 |
| 287T > C | I96T | Missense | PAS-domain | Da | Larsen et al, 2001 |
| 296A > C | Y99S | Missense | PAS domain | Du | Jongbloed et al, 2002 |
| 391A > G | K101E | Missense | PAS-domain | Da | Larsen et al, 2001 |
| — | M124R | Missense | — | USA | Moss et al, 2002 |
| 422insC | 1 bp insertion | Frameshift | PAS-S1 | USA | Splawski et al, 2000 |
| 453insC | 1 bp insertion | Frameshift | PAS-S1 | USA | Splawski et al, 2000 |
| 453delC | 1 bp deletion | Frameshift | PAS-S1 | Fi | Swan et al, 1999, |
| | | | | | Lätinen et al, 2000 |
| 526C > T | R176W | Missense | PAS-S1 | Fi | Swan et al, 1999, |
| | | | | | Lätinen et al, 2000 |
| 558-600dup | 42 bp duplication | Frameshift | PAS-S1 | Mediter-ranean | Hoorntje et al, 1999 |
| 582-587 delCCGTG | G192fs328 | Deletion | PAS-S1 | Du | Jongbloed et al, 2002 |
| 724insC | 1 bp insertion | Frameshift | PAS-S1 | USA | Splawski et al, 2000 |
| 885delC | 1 bp insertion | Frameshift | PAS-S1 | USA | Splawski et al, 2000 |
| — | fsV295/63aa | Frameshift | PAS-S1 | USA | Moss et al, 2002 |
| 934C > T | R312C | Missense | PAS-S1 | USA | Splawski et al, 2000 |
| 1039G > A | P347S | Missense | PAS-S1 | USA | Splawski et al, 2000 |
| 1039C > T | P347S | Missense | PAS-S1 | Du | Jongbloed et al, 2002 |
| 1096C > T | R366X | Nonsense | PAS-S1 | Da | Larsen et al, 2001 |
| — | Q376- | Splice site | PAS-S1 | USA | Moss et al, 2002 |
| 1128G > A | — | Splice site | PAS-S1 | USA | Splawski et al, 2000 |
| 1129-2 G > A | — | Splice site | PAS-S1 | USA | Splawski et al, 2000 |
| 1199T > A | I100N | Missense | S1 | Da | Larsen et al, 2001 |
| 1261delA | 1 bp deletion | Frameshift | S1 | It | Curran et al, 1995 |
| 1283C > A | S428X | Nonsense | S1-S2 | It | Priori et al, 1999 |
| — | S428L | Missense | S1-S2 | USA | Moss et al, 2002 |
| 1307C > T | T436M | Missense | S1-S2 | It | Priori et al, 1999 |
| 1341C > A | Y447X | Nonsense | S1-S2 | Du | Jongbloed et al, 2002 |
| 1352C > T | P451L | Missense | S2 | Fi | Lä\ddot{ }tinen et al, 2000 |
| 1408A > G | N470D | Missense | S2 | Me, Ge, Eng, Da | Curran et al, 1995 |
| 1421C > T | T474I | Missense | S2-S3 | Ja | Tanaka et al, 1997 |
| 1479C > G | Y493X | Nonsense | S2-S3 | Ja | Itoh et al, 1998 |
| 1496-1523dle | I500-F508del | Deletion | S3 | Me, Ge, Eng, Da | Curran et al, 1995 |
| 1501A > G | D501N | Missense | S3 | Du | Jongbloed et al, 2002 |
| 1592G > A | R531Q | Missense | S3 | USA | Splawski et al, 2000 |
| — | L552S | Missense | S3 | USA | Moss et al, 2002 |
| 1600C > T | R635C | Missense | S4 | Ja | Itoh et al, 1998, |
| | | | | | Nakajima et al, 1999 |
| 1631delAG | 2 bp deletion | frameshift | S4-S5 | Fi | Wsan et al, 1999, |
| | | | | | Lätinen et al, 2000 |
| 1655T > C | L552S | Missense | S5 | Fi, USA | Swan et al, 1999, |
| | | | | | Splawski et al, 2000 |
| 1672G > C | A558P | Missense | S5 | Du | Jongbloed et al, 1999 |
| 1681G > A | A561T | Missense | S5 | Fr, USA | Dausse et al, 1996, |
| | | | | | Splawski et al, 2000 |
| | | | | | Curran et al, 1995, |
| | | | | | Tanaka et al, 1997, |
| 1682C > T | A561V | Missense | S5 | Me, Ge, Emg, Da, Ja, | Priori et al, 1999, |
| | | | | USA | Larsen et al, |
| | | | | | unpublished, |
| | | | | | Splawski et al, 2000 |
| 1691T > C | L564P | Missense | S5 | Fr, Ca | St-Pierre et al, 2000 |
| 1705T > C | Y569H | Missense | S5 | Fi | Swan et al, 1999, |
| | | | | | Lätinen et al, 2000 |
| 1714G > C | G572R | Missense | S5-pore | Da | Larsen et al, 2000 |
| 1714G > T | G572C | Missense | S5-pore | Du | Jongbloed et al, 1999 |
| | | | | | swan et al, 1999, |
| 1750G > A | W585C | Missense | S5-pore | Fi, USA | Splawski et al, 2000, |
| | | | | | Lätinen et al, 2000 |
| 1755G > T | W585C | Missense | S5-pore | USA | Splawski et al, 2000 |
| 1762A > G | N588D | Missense | S5-pore | USA | Splawski et al, 2000 |
| 1778T > C | I593R | Missense | S5-pore | Eng?, USA | Benson et al, 1996, |
| | | | | | Splawski et al, 2000 |
| 1778T > G | I593G | Missense | S5-pore | USA | Benson et al, 1996, |
| | | | | | Splawski et al, 2000 |
| — | I593X | Nonsense | S5-pore | USA | Moss et al, 2002 |
| — | P596L | Missense | S5-pore | USA | Moss et al, 2002 |
| | | | | | Akimoto et al, 1997, |
| 1801G > A | G601S | Missense | S5-pore | Ja, Fi | Swan et al, 1999, |
| | | | | | Lätinen et al, 2000 |
| 1810G > A | G604S | Missense | S5-pore | Du, USA | Jongbloed et al, 1999, |
| | | | | | Splawski et al, 2000 |
| 1825G > A | D609N | Missense | S5-pore | USA | Splawski et al, 2000 |
| 1831T > C | Y611H | Missense | S5-pore | Ja | Tanaka et al, 1997 |
| 18331 > A/G | Y611X | Nonsense | S5-pore | Ge | Schultze-Bahr et al, 1995 |
| 1834G > T | V612L | Missense | S5-pore | Ir, It, Du | Satler et al, 1998 |
| 1838C > T | T613M | Missense | S5-pore | Du, Fi | Jongbloed et al, 1999, |
| | | | | | Lätinen et al, 2000 |
| | | | | | Tanaka et al, 1997, |
| 1842C > T | A614V | Missense | Pore | Ja, Ir, Cz, Eng, Fr, Fi, | Satler et al, 1998, |
| | | | | It, Tw | Splawski er al, 1998, |
| | | | | | Priori et al, 1999, |
| | | | | | Lee-Chen et al, 1999 |
| 1843G > | L615V | Missense | Pore | USA | Splawski et al, 2000 |
| 1862G > A | S621N | Missense | Pore | Da | Larsen et al, 2001 |
| 1876G > A | G626S | Missense | Pore | USA | Splawski et al, 2000 |
| 1877G > T | G626V | Missense | Pore | Ge | Jahr et al, 2000 |
| 1881G > C | F627L | Missense | Pore | USA | Splawski et al, 2000 |
| 1882G > A | G628S | Missense | Pore | Me, Ge, Eng, Da | Curran et al, 1995, |
| | | | | | Splawski et al, 2000 |
| | | | | | Satler et al, 1998, |
| 1885A > G | N629D | Missense | Pore | Ge, Ir, Na | Lees-Miller et al, 2000 |
| | | | | | Satler et al, 1998, |
| 1886A > G | N629S | Missense | Pore | Unknown, Da | Larsen et al, |
| | | | | | unpublished |
| — | N629K | Missense | Pore | Ja | Yoshida et al, 1999 |
| 1888G > C | V630L | Missense | Pore | Ja | Tanaka et al, 1997 |
| 1889T > C | V630A | Missense | Pore | USA | Splawski et al, 1998 |
| 1894C > T | P632S | Missense | Pore | USA | Splawski et al, 2000 |
| 1898A > G | N633S | Missense | Pore | Eng | Satler et al, 1998 |
| 1912A > G | K638E | Missense | S6 | USA | Splawski et al, 2000 |
| 1913-15del | K638del | Deletion | S6 | USA | Splawski et al, 2000 |
| 1920C > A | F640L | Missense | S6 | Du | Jongbloed et al, 1999 |
| 1933A > T | M645L | Missense | S6 | USA | Splawski et al, 2000 |
| 1951-1952delAT | 2 bp deletion | Frameshift | S6 | Ja | Itoh et al, 1998 |
| 2001C > A | Y667X | Nonsense | S6 | Be | Paulussen et al, 2000 |
| 2044G > T | E682X | Nonsense | S6 | USA | Splawski et al, 2000 |
| 2092G > T | E698X | Nonsense | S6 | Du | Jongbloed et al, 2002 |
| 2173C > T | Q725X | Nonsense | S6 | Ja | Itoh et al, 1998 |
| 2218insT | 1 bp insertion | Frameshift | S6-- | USA | Splawski et al, 2000 |
| 2232C > T | R744X | Nonsense | S6-- | Tw | Ko et al, 2001 |
| 2254C > T | R752W | Nonsense | S6-- | USA | Splawski et al, 2000, |
| | | | | | Ficher e al, 2000 |
| 2350C > T | R784W | Missense | S6-- | USA | Yang et al, 2002 |
| 2356-2376dup | 31 bp duplication | Frameshift | CNB domain | Ja | Itoh et al, 1998 |
| 2395delC | 1 bp deletion | Frameshift | CNB domain | USA | Splawski et al, 2000 |
| 2398 + 1G > C | IVS9 DS + 1 | Splice mutation | CNB domain | USA | Curran et al, 1995 |
| 2414T > C | F805S | Missense | CNB domain | USA | Splawski et al, 2000 |
| 2414T > G | F805C | Missense | CNB domain | USA | Splawski et al, 2000 |
| 2453C > T | S818L | Missense | CNB domain | Fr | Berthet et al, 1999 |
| 2464G > A | V822M | Missense | CNB domain | Ir, Fr | Satler et al, 1996, |
| 2467C > T | R823W | Missense | CNB domain | USA | Berthet et al, 1999 |
| | | | | | Splawski et al, 2000 |
| 2471insG | R823fs828 | Deletion | CNB domain | Du | Jongbloed et al, 2002 |
| 2582A > T | N861I | Missense | C-terminal | USA | Splawski et al, 2000 |
| | | | | | Berthet et al, 1999 |
| 2592 + 1G > A | IVS1 + DS + 1 | Frameshift | C-terminal | Be, USA | |
| | | | | | Splawski et al, 2000 |
| 2616delC | P872fs877 | Deletion | C-terminal | Du | Jongbloed et al, 2002 |
| 2660delG | 1 bp deletion | Frameshift | C-terminal | USA | Splawski et al, 2000 |
| 2750C > T | P917L | Missense | C-terminal | USA | Splawski et al, 2000 |
| 2762delA | 1 bp deletion | Frameshift | C-terminal | USA | Splawski et al, 2000 |
| 2764C > T | R922W | Missense | C-terminal | USA | Splawski et al, 2000 |
| 2775insG | 1 bp insertion | Frameshift | C-terminal | USA | Splawski et al, 2000 |
| 2906delG | 1 bp deletion | Frameshift | C-terminal | USA | Splawski et al, 2000 |
| — | P968/4aa | Frameshift | C-terminal | USA | Moss et al, 2002 |
| 2959delCT | 2 bp deletion | Frameshift | C-terminal | USA | Splawski et al, 2000 |
| — | W1001X | Nonsense | C-terminal | USA | Moss et al, 2002 |
| 3040C > T | R1014X | Nonsense | C-terminal | USA | Splawski et al, 2000 |
| 3094delC | 1 bp deletion | Frameshift | C-terminal | USA | Splawski et al, 2000 |
| 3108insG | — | Frameshift | C-terminal | Sp | Berthet et al, 1999 |
| 3303insC | 1 bp insertion | Frameshift | C-terminal | USA | Splawski et al, 2000 |
| | | | | | Iwasa et al, 2000, |
| 2690A > C | K897T | — | — | — | Lätinen et al, 2000, |
| | | | | | Yang et al, 2002 |
| — | R1047L | — | — | — | Larsen et al, 2001 |
| IVS2 + 27G > C | Intron variant | SNP | — | — | Jongbloed et al, 2002 |
| IVS8-61 A/G | Intron variant | SNP | — | — | Larsen et al, 2001 |
| IVS13 + 12 C/A | Intron variant | SNP | — | — | Larsen et al, 2001 |
| IVS13 + 22 A/G | Intron variant | SNP | — | — | Larsen et al, 2001 |
| 1467C > T | I489I | SNP | — | — | Akimoto et al 1997 |
| 1539C > T | F513F | SNP | — | — | Akimoto et al, 1997 |
| 1692A > G | L564L | SNP | — | — | Akimoto et al, 1997 |
| 1956T > C | Y652Y | SNP | — | — | Larsen et al, 1999 |
| 2965+22 A > G | Intronic | — | — | — | Iwasa et al, 2000 |
| | variant | | | | |

¹ Ja: Japanese: Ir: Irish, Cz: Czech, Eng: English; Ge: German; Fr: French; Fil: Filipino; Fi: Finnish; Na: Native American; Me: Mexican; Da: Danish; It: Italian; Du: Dutch; Sp: Spanish; Be: Belgian; Tw: Taiwanese.
² Region distal to S6.

The inventors also contemplate that the cells can express mutant hERG 1b subunits. As shown below in the Examples, mutations in the hERG 1b retention signal promote hERG 1b maturation.

Any of the contemplated polynucleotides can be cloned into an expression vector (or plurality of expression vectors) engineered to support expression of the polynucleotides. Suitable expression vectors comprise a transcriptional promoter active in a recipient cell upstream of the hERG1 polynucleotide and can optionally comprise a polyA-addition sequence downstream of the polynucleotide. Optionally, an expression vector can comprise a selectable marker to Facilitate selection of those cells that carry the vector.

Suitable commercially available expression vectors are pcDNA3.1 and pcDNA3.1zeo (Invitrogen Corp.; Carlsbad, Calif.), which differ from one another in that pcDNA3.1 includes sequences conferring resistance to neomycin while pcDNA3.1 zeo includes sequences conferring resistance to zeocin. The polyA-addition sequences, not required for expression, can be excised from these vectors by digesting both with ApaI (nuc. 1002) and BbsI (nuc. 1217), respectively, filling in and self-ligating. The vectors can be prepared to receive hERG 1b, and optionally hERG 1a, polynucleotides, by cleavage with EcoRI and BamHI. For convenience during the subsequent selection step, hERG1b can then be cloned into the cleaved pcDNA3.1 vector; a mutant hERG 1a can be cloned into the cleaved pcDNA3.1zeo vector. In addition, hERG 1b and mutant hERG 1a polynucleotides can be ligated into the two multiple cloning sites of a vector with an internal ribosomal entry site, such as pIRES (Novagen), which allows for production of two separate proteins from a single transcript. Use of this vector to produce cells allows for selection with a single antibiotic. Other suitable vectors for use herein include pGH19 and pEGFP-N1.

The vector(s) can be introduced (or co-introduced) by, for example, transfection or lipofection, into cells competent to receive and express such vector. A commercially available lipofection kit, such as a kit available from Mirus Corporation (Madison, Wis.) can be employed. Advantageously, the recipient cells lack endogenous hERG 1b, so that the formation of hERG 1b-containing channels is attributable to expression of the introduced expression vector. Suitable recipient cells are described above.

After introduction of the expression vector, preferably about twenty-four to forty-eight hours after introduction, the cells can be harvested, distributed into wells, and grown in selective media. In the exemplified embodiment, a medium suitable for selecting cells carrying the hERG 1b-vector contains neomycin at 500 μg/ml, a medium suited for carrying the hERG 1a-vector (contains zeocin at 100 μg/ml, and a medium suited for growing cells carrying both vectors contains both antibiotics. Cells can be grown under selection for 2-3 weeks until the wells are confluent. Resulting cells (24-48 for each type) can be examined biochemically or electrophysiologically to confirm the presence of the hERG1 channel subunit(s) and to determine the level(s) of hERG produced.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

As used herein, a “coding sequence” means a sequence that encodes a particular polypeptide, and is a nucleic acid sequence transcribed (in the case of DNA) and translated (in the case of mRNA) into a polypeptide in vitro or in vivo when placed under the control of appropriate regulatory sequences. The boundaries of the coding sequence are determined by a start codon at a 5′ (amino) terminus and a translation stop codon at a 3′ (carboxy) terminus. A coding sequence can include, but is not limited to, viral nucleic acid sequences, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, an “expression sequence” means a control sequence operably-linked to a coding sequence.

As used herein, “control sequences” mean promoter sequences, polyadenylation signals, transcription termination sequences, upstream regulatory domains, origins of replication, internal ribosome entry sites (“IRES”), enhancers, and the like, which collectively provide for replication, transcription and translation of a coding sequence in a recipient cell. Not all of these control sequences need always be present so long as the selected coding sequence is capable of being replicated, transcribed and translated in an appropriate cell.

As used herein, a “promoter” means a nucleotide region comprising a nucleic acid (i.e., DNA) regulatory sequence, wherein the regulatory sequence is derived from a gene capable of binding RNA polymerase and initiating transcription of a downstream (3′-direction) coding sequence. Transcription promoters can include “inducible promoters” (where expression of a polynucleotide sequence operably-linked to the promoter is induced by an analyte, cofactor, regulatory protein, etc.), “repressible promoters” (where expression of a polynucleotide sequence operably-linked to the promoter is repressed by an analyte, cofactor, regulatory protein, etc.) and “constitutive promoters” (where expression of a polynucleotide sequence operably-linked to the promoter is unregulated and therefore continuous).

As used herein, a “nucleic acid sequence” or “polynucleotide” means a DNA or RNA sequence. The term encompasses sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine and 2,6-diaminopurine.

As used herein, “operably-linked” means that elements of an expression sequence are configured so as to perform their usual function. Thus, control sequences (i.e., promoters) operably-linked to a coding sequence are capable of effecting expression of the coding sequence. The control sequences need not be contiguous with the coding sequence, so long as they function to direct the expression thereof. Thus, for example, intervening untranslated, yet transcribed, sequences can be present between a promoter and a coding sequence, and the promoter sequence can still be considered “operably-linked” to the coding sequence.

As used herein, “operable interaction” means that subunits of a polypeptide, and any other accessory proteins, that are heterologously expressed in a cell assemble into a functioning hERG channel with potassium conductance.

A “vector” is a replicon, such as a plasmid, phage, or cosmid, to which another nucleic ac id segment may be attached so as to bring about the replication of the attached segment. A vector is capable of transferring gene sequences to target cells (e.g., bacterial plasmid vectors, particulate carriers, and liposomes).

Typically, the terms “vector construct,” “expression vector,” “gene expression vector,” “gene delivery vector,” “gene transfer vector,” and “expression cassette” all refer to an assembly capable of directing the expression of a sequence or gene of interest. Thus, the terms include cloning and expression vehicles.

As used herein, “endogenous” means inherent to the unaltered cell.

As used herein, “hERG 1b N-terminus” means the amino-terminal portion of the hERG 1b protein encoded by the hERG 1b-specific exon.

The invention will be more fully understood upon consideration of the following non-limiting Examples.

### EXAMPLES

### Example 1

### hERG 1b Amino-Terminal Signal Prevents Maturation

Methods: Reagents, antibodies, cell transfection, lysis, co-immunoprecipitation and Western blot analysis were performed as described by Phartiyal et al. Phartiyal P, et al., “Heteromeric assembly of human ether-a-go-go-related gene (hERG) 1a/1b channels occurs cotranslationally via 3N-terminal interactions,” J. Biol. Chem. 282:9874-9882 (2007); and Phartiyal P, et al., “Endoplasmic reticulum retention and rescue by heteromeric assembly regulate human ERG 1a/1b surface channel composition,” J, Biol, Chem. 283:3702-3707 (2008), each of which is incorporated herein by reference as if set forth in its entirety.

Whole cell patch clamping: Wild-type hERG 1b and mutant hERG 1b were cloned into a pGH19 expression vector (see, Liman E, et al., “Voltage-sensing residues in the S4 region of a mammalian K+ channel,” Nature 353:752-756 (1991), incorporated herein by reference as if set forth in its entirety) or into a pEGFP-N1 expression vector (Clonetech Laboratories, Inc.; Mountain View, Calif.) as described by Trudeau et al. Trudeau M, et al., “HERG, a human inward rectifier in the voltage-gated potassium channel family,” Science 269:92-95 (1995), incorporated herein by reference as if set forth in its entirety. Approximately 1.5 mg of wild-type hERG 1b or mutant hERG 1b cDNA was transiently or stably expressed in Xenopus oocytes, COS-7 cells (ATCC Accession No. CRL-1651) or HEK-293 cells (ATCC Accession Catalog No. CRL-1573). When pEGFP-N1 was used, fluorescent cells were chosen for electrical recordings. Whole-cell patch clamp recordings were performed using an Axopatch 200B amplifier (Molecular Devices; Sunnyvale, Calif.). A bath solution contained the following: 137 mM NaCl, 4 mM KCl, 1.8 mM CaCl₂, 1 mM MgCl₂, 10 mM glucose, 5 mM tetraethyl ammonium (TEA) and 10 mM HEPES (pH 7.4 with NaOH). TEA was included in the bath solution to block endogenous, voltage-gated, potassium channels. Whole-cell currents were recorded with a fire-polished pipette tip of approximately 1-2 mm with a resistance of 2-4 MΩ. An internal pipette solution contained the following: 130 mM KCl, 1 mM MgCl₂, 5 mM EGTA, 5 mM MgATP, 10 mM HEPES (pH 7.2 with KOH). Currents were digitized at 2 kHz unfiltered. Series resistance compensation was typically 60% to 70%, such that voltage errors were less than 5 mV. Leak subtraction was not used. Current recordings were carried out at room temperature (i.e. ˜18° C. to 23° C.).

Two-electrode voltage clamping in oocytes: Two-electrode voltage clamping in oocytes, cRNA preparation and injection were performed as previously described by Wang et al. Wang J, et al., “Regulation of deactivation by an amino terminal domain in human ether-a-go-go-related gene potassium channels,” J Gen Physiol. 112:637-647 (1998), incorporated herein by reference as if set forth in its entirety.

hERG 1b mutations: A scan of the amino terminus of ERG 1b in various mammals (i.e., human, mouse, rat, and dog) revealed two RXR motifs in ERG 1b (Table 2). In hERG 1b, the first RXR motif begins at amino acid 15 (R₁₅XR) and the second RXR motif begins at amino acid 22 (R₂₂XR). Only R₂₂XR was conserved across ERG in the various species, as R₁₅XR is replaced by QXR. Various deletions or mutations were introduced into the hERG 1b gene.

TABLE 2
RXR Motifs in ERG 1b.

| ERG 1b | Sequence |
|---|---|
| hERG 1b | MAAPAGKASRTGALRPRAQKGRVRRAVRISSLVAQE |
| | (SEQ ID NO: 3) |
| mERG 1b | MAIPAGKESRTGALQPRAQKGRVRRAVRISSLVAQE |
| | (SEQ ID NO: 4) |
| rERG 1b | MAIPAGKESRTGALQTRAQKGRVRRAVRISSLVAQE |
| | (SEQ ID NO: 5) |
| cERG 1b | MAAPAGKASRTGALQPRAQKGRVRRAVRISSLVAQE |
| | (SEQ ID NO: 6) |

Results: Deletion of amino acids 2-17 (hERG 1bΔ2-17) or the R₁₅XR motif in hERG 1b facilitated maturation in HEK-293 cells as detected by Western blotting, which served as a proxy for surface expression. In fact, hERG 1b maturation increased about 2.6 fold, and was similar to that observed when wild-type hERG 1b was co-expressed with hERG 1a. This deletion also stabilized expression of hERG 1 bA2-17 protein in the plasma membrane at a level comparable to that of wild type hERG 1b in the presence of hERG 1a.

The R₁₅XR motif, but not R₂₂XR, caused hERG 1b retention in the endoplasmic reticulum. Mutation of the arginines in R₂₂XR to asparagines (N₂₂XN) in Xenopus oocytes showed no significant increase in maturation over wild-type hERG 1b. In contrast hERG 1b in which the arginines in R₁₅XR were mutated to lysines (K₁₅XK, conserved charge), asparagines (N₁₅XN, neutral) or aspartates (D₁₅XD, reversed, negative charge) exhibited increased maturation relative to wild-type hERG 1b. Compared to wild-type hERG 1b, K₁₅XK hERG 1b demonstrated a slightly improved maturation; whereas N₁₅XN hERG 1b and D₁₅XD hERG 1b progressively enhanced hERG 1b maturation up to approximately five-fold. Thus, the increased maturation of the R₁₅XR mutants was charge-dependent. A double mutation of both RXR (N₁₅XN/N₂₂XN) motifs in hERG 1b, however, did not improve maturation over N₁₅XN.

Mutant hERG 1b showed functional expression at a cell surface. Currents from Xenopus oocytes injected with cDNA from K₁₅XK, N₁₅XN or D₁₅XD hERG 1b showed currents up to about four-fold higher than wild-type hERG 1b in a charge-dependent manner, with D₁₅XD hERG 1b showing the strongest currents. Compared to wild-type hERG 1b, the mutants show no differences in channel kinetics or in voltage dependence of currents, which suggested that larger currents were due to increased channel number at the cell surface and were not due to altered gating properties. More importantly, HERG 1b subunits having N₁₅XN and D₁₅XD form homomeric channels that produce potassium current in the absence of hERG 1a or any other accessory subunit, if not prevented from trafficking to the cell surface.

Wild-type and mutant hERG 1b localized to distinct cell structures. GFP-tagged wild-type hERG 1b was retained in perinuclear compartments in COS-7 cells. In contrast, GFP-tagged N₁₅XN hERG 1b and GFP-tagged wild-type hERG 1b co-expressed with hERG 1a were both localized away from perinuclear compartments. Approximately twice as many cells had a diffuse, non-perinuclear signal for GFP-tagged N₁₅XN hERG 1b and GFP-tagged wild-type hERG 1b co-expressed with hERG 1a when compared to GFP-tagged hERG 1b alone. Similar results were obtained with HEK-293 cells.

### Example 2

### hERG 1a Rescues hERG 1b Surface Expression by Masking an Endoplasmic Reticulum Retention Signal

Methods: Reagents, antibodies, cell transfection, lysis, co-immunoprecipitation, patch clamping, mutant hERG 1b and Western blot analysis were performed as described above.

Cells: Cell lines used in this example are described above.

Two-electrode voltage clamping was performed as described above.

The effect of hERG 1a on hERG 1b maturation was examined in Xenopus oocytes. Briefly, hERG 1b and N₁₅XN hERG 1b cDNA were cloned into a pGH19 expression vector as described above. cRNA transcripts were purified through a G-50 column (Amersham Biosciences; Piscataway, N.J.), and 30 ng cRNA was injected into Xenopus oocytes. Currents were recorded two to three days after injection using a two electrode voltage clamp. Oocytes were held at a potential of −80 mV. Currents were evoked by 480 ms pulses ranging from −80 to +40 mV in increments of +20 mV, followed by a repolarizing step to −105 mV for 480 ms.

Results: hERG 1a facilitated maturation of hERG 1b. When expressed alone, hERG 1b showed negligible maturation. However, in the presence of hERG 1a, hERG 1b maturation increased nearly three-fold by Western blot analysis. In addition, hERG 1b protein increased about ten-fold when hERG 1a was present. hERG 1a therefore facilitated endoplasmic reticulum exit and Golgi maturation of hERG 1b subunits. In addition, the increased maturation noted above in Example 1 for N₁₅XN hERG 1b was not enhanced in the presence of hERG 1a. Thus, hERG 1a and mutant hERG 1b appear to facilitate maturation by the same or related mechanisms. N₁₅XN hERG 1b co-precipitated with hERG 1a in presence of a hERG 1a-specific antibody, suggesting that its failure to mature was not caused by a failure to associate with hERG 1a. Instead, hERG 1a may facilitate maturation by masking the hERG 1b RXR retention signal.

### Example 3

### Identification of Agents that Facilitate hERG 1b Maturation in Mammalian Cells

Methods: Cell transfection is performed as described above.

Cells: The cell lines used in this example are described above. Briefly, HEK-293 cells are stably transfected with a GFP-tagged hERG 1b expression vector as described in Example 1.

Screening assay: Transfected HEK-293 cells are plated in minimum essential medium supplemented with 10% FBS, 1% non-essential amino acids, 1% sodium pyruvate, and 400 μg/ml geniticin. To test an agent for its ability to facilitate hERG 1b maturation, the agent is added to the medium. Following incubation, the cells are trypsinized, washed in standard minimal essential medium, and plated onto cover slips for visualization by fluorescent microscopy.

Results: In the presence of an agent that facilitates hERG 1b maturation, hERG 1b is localized away from the perinuclear compartment creating a diffuse, non-perinuclear hERG 1b signal. In contrast, if an agent does not facilitate maturation of hERG 1b, hERG 1b is localized in the perinuclear compartment creating a perinuclear hERG 1b signal.

### Example 4

### Mutagenesis of hERG 1b

Methods: The hERG 1b-specific exon encoding for the N-terminal cytoplasmic domain was mutated using polymerase chain reaction (PCR), denaturing high performance liquid chromatography (DHPLC), and direct DNA sequencing as described by Tester et al. Tester D, et al., “Mutation detection in congenital long QT syndrome: cardiac channel gene screen using PCR, dHPLC, and direct DNA sequencing,” Methods Mol. Med. 128:181-207 (2006), incorporated herein by reference as if set forth in its entirety. Previously published PCR primers and reaction conditions were used in this study. Splawski I, et al., “Genomic structure of three long QT syndrome genes: KVLQT1, hERG, and KCNE1,” Genomics 51 (1):86-97 (1998), incorporated herein by reference as if set forth in its entirety. An A8V mutation was introduced into a hERG 1b sequence using a PCR-based mutagenesis strategy. Sale H, et al., “Physiological Properties of hERG 1a/1b heteromeric currents and a hERG 1b-specific mutation associated with long-QT syndrome,” Circulation Research 103:e81-e95 (2008), incorporated herein by reference as if set forth in its entirety. Subsequent sequence analysis confirmed the presence of the mutation and the absence of any other hERG 1b sequence alterations. Western blots were prepared following standard procedures. Briefly, HEK-293 cells were transfected with 1.5 μg of each DNA construct using LT1 reagent (Mirus). Cells were lysed forty-eight hours post-transfection and processed as described above in Example 1. The cell lysate (5-10 μg) was separated by SDS-PAGE, transferred to Immobilon-P polyvinylide difluoride membranes (Millipore, Bedford, Mass.) and probed with antibodies specifically recognizing the C-terminus of hERG. Roti Roti E, et al., “Interaction with GM130 during HERG ion channel trafficking. Disruption by type 2 congenital long QT syndrome mutations,” J. Biol. Chem. 277:47779-47785 (2002). Membranes were also probed with antibodies specific to protein disulfide isomerase, a protein abundant in the endoplasmatic reticulum, as a loading control.

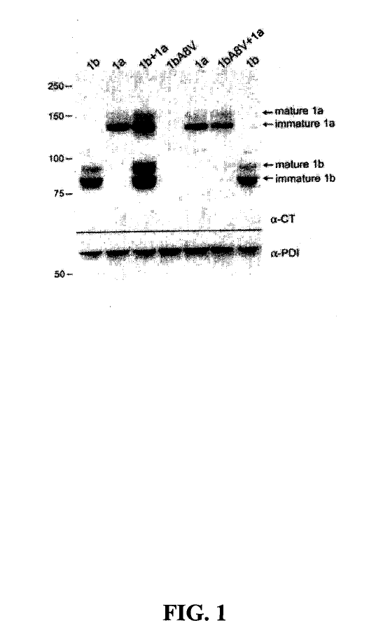

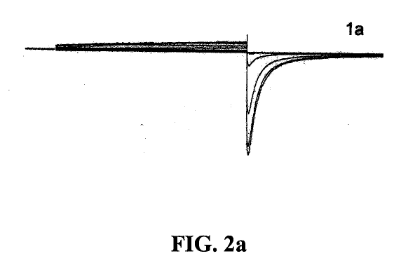

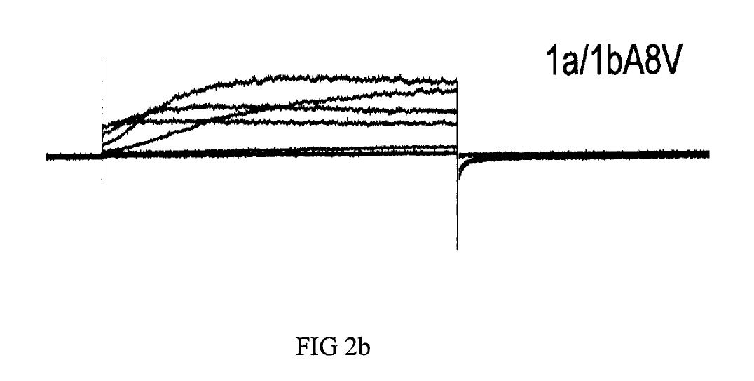

Results: Western blot analysis of cell lysates from HEK-293 cells transfected with a A8V-hERG 1b gene construct showed a dramatic reduction in mature and immature hERG 1b protein when compared to cells transfected with the wild type hERG 1b gene (FIG. 1, compare lane 4 to lanes 1 and 7). This reduction was only slightly mitigated by co-expression with hERG 1a (FIG. 1, lane 6). Expression of A8V-hERG 1b in hERG 1a-expressing cells reduced total conductance, as evident from the tail current amplitudes, and reduced rectification, as evident from the ratio of the current amplitude at the end of the test pulse relative to the tail current amplitude (FIG. 2B, compared to FIG. 2A). While these results indicate that some hERG 1a/1b heteromers are formed, the dramatic reduction in hERG 1b protein caused by the A8V mutation is expected to decrease the number of these heteromers and thus reduce the contribution of Iₖᵣ to ventricular repolarization. The presence of the A8V mutation, thus, predicts a loss of function in individuals carrying this mutation.

### Example 5

### Identifying Mutations in the hERG 1b-Specific Exon Associated with Arrhythmia (Prophetic)

DNA and/or protein from individuals afflicted with arrhythmia is screened using standard nucleotide and protein analysis for alterations in the hERG 1b-specific nucleotide and/or amino acid sequence. Alterations in the hERG 1b-specific nucleotide and/or amino acid sequence can be analyzed further in vitro for their effect on hERG 1b protein function and hERG 1a/1b channel formation.

### Example 6

### Identifying Individuals at Risk for Arrhythmia (Prophetic)

DNA and/or protein is isolated from an individual in need of receiving or scheduled to receive drugs that can block hERG channels. The DNA is analyzed by standard methodology to determine if the individual harbors a mutation in the hERG 1b-specific exon, which encodes the hERG 1b N-terminal cytoplasmic domain, that alters the hERG 1b protein amino acid composition. Alternatively or additionally, the protein is analyzed to determine if the individual harbors an hERG 1b N-terminal cytoplasmic domain amino acid sequence different from the amino acid sequence of SEQ ID NO. 2.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

The invention claimed is:

1. A cell engineered for screening for agents that increase maturation of human ether-a-go-go-related gene (hERG) 1b, the cell comprising:
a polynucleotide encoding a polypeptide (SEQ ID NO:2) for hERG 1b operably-linked to an upstream expression control sequence not endogenously linked to the polynucleotide, wherein in the cell does not endogenously express hERG channels, and wherein the encoded hERG 1b is localized in a perinuclear compartment.

2. A cell as recited in claim 1, wherein the cell is a member selected from the group consisting of a Xenopus oocyte, a CHO-K1 cell, a L929 cell, a HEK-293 cell and a COS-7 cell, which is engineered to comprise the polynucleotide.

3. A cell as recited in claim 1, wherein the hERG 1b is further operably-linked to a reporter.

4. A cell as recited in claim 3, wherein the reporter is selected from a FLAG tag, a GST-tag, a His-tag, a c-myc tag, a hemagglutinin tag and green fluorescent protein or a fluorescene derivative thereof.

5. A cell as recited in claim 1, wherein the polynucleotide comprises SEQ ID NO:1.

6. A method of identifying an agent that facilitates human ether-a-go-go-related gene (hERG) 1b maturation, the method comprising the steps of:
exposing a cell comprising a polynucleotide encoding a polypeptide (SEQ ID NO:2) for hERG 1b operably-linked to an upstream expression control sequence not endogenously linked to the polynucleotide to a test agent suspected of promoting hERG 1b maturation, wherein the cell does not endogenously express hERG channels, and wherein hERG 1b is localized in a perinuclear compartment; and
evaluating localization of hERG 1b in the cell exposed to the test agent relative to the cell not exposed to the test agent,
whereby the test agent facilitates maturation if the hERG 1b was trafficked to a non-perinuclear compartment.

7. A method as recited in claim 6, wherein the polynucleotide is further operably-linked to a reporter.

8. A method as recited in claim 6, wherein the cell is a member selected from the group consisting of a Xenopus oocyte, a CHO-K1 cell, a L929 cell, a HEK-293 cell and a COS-7 cell, which is engineered to comprise the polynucleotide.

9. A method as recited in claim 7, wherein the reporter is selected from a FLAG tag, a GST-tag, a His-tag, a c-myc tag, a hemagglutinin tag and green fluorescent protein or a fluorescene derivative thereof.

10. A method as recited in claim 6, wherein the polynucleotide comprises SEQ ID NO:1.

11. A method for analyzing a biological sample for the presence of an amino acid substitution at position 8 of the hERG 1b N terminus, the method comprising the steps of:
(a) obtaining the biological sample
(b) analyzing the sample for the presence of an amino acid substitution at position 8 of the hERG 1b N terminus; and
(c) detecting the presence of an amino acid substitution at amino acid position 8 of the hERG 1b N terminus.

12. The method of claim 11, wherein the substitution is an alanine-to-valine substitution.

13. The method of claim 11, wherein the sample is analyzed using nucleotide analysis.

14. The method of claim 13, wherein the sample is analyzed using nucleotide sequencing.

15. The method of claim 13, wherein the sample is analyzed using array-based hybridization.

16. The method of claim 11, wherein the sample is analyzed using amino acid analysis.
